(19) United States
(12) Patent Application Publication
(10) Pub. No.: US 2004/0219097 A1
(43) Pub. Date: Nov. 4, 2004

(54) Composition useful for the diagnosis, imaging and treatment of tumors

(76) Inventor: Jasbir Sandhu, Burlington (CA)

Correspondence Address: Michael A. Slavin, Esq., McHale & Slavin, P.A., Suite 402, 4440 PGA Boulevard, Palm Beach Gardens, FL, 33410, US

(21) Appl. No.: 10/429,482

(22) Filed: May 2, 2003

(51) Int. Cl. A61K 51/00

(52) U.S. Cl. 424/1.69; 530/400

(57) ABSTRACT

The invention provides a composition containing human vascular endothelial growth factor (VEGF), radiolabeled human transferrin and human epidermal growth factor (EGF). The composition is useful for the diagnosis, imaging and treatment of tumors and other diseases.

### CROSS REFERENCE TO RELATED APPLICATIONS

The instant application is related to application Ser. Nos. 10/______; 10/______; 10/______; 10/______; 10/______; 10/______; 10/______; 10/______; 10/______; 10/______ and 10/______; all filed on even date herewith under Express Mail labels EV 001630864 US; EV 140261673 US; EV 140261660 US; EV 140261585 US; EV 140261571 US; EV 140261568 US; EV 140261554 US; EV 140261537 US; EV 140261523 US; EV 001630855 US and EV 001630847 US; the contents of which are each herein incorporated by reference.

### FIELD OF THE INVENTION

The instant invention relates generally to a composition useful in the diagnosis, imaging and treatment of cancer and other diseases; particularly to a composition useful for a multi-targeted approach to cancer diagnosis, imaging and treatment and most particularly to a composition containing human vascular endothelial growth factor (VEGF) and human epidermal growth factor (EGF) operatively linked to radiolabeled human transferrin useful for a multi-targeted approach to cancer diagnosis, imaging and treatment.

### BACKGROUND OF THE INVENTION

Malignant disease is a major cause of mortality and morbidity in most countries. Despite the impressive advances in scientific knowledge and improved therapy of malignant disease, many prevalent forms of human cancer still resist effective therapeutic intervention. Current diagnostic and therapeutic methods remain ineffective. The treatment of metastatic disease remains particularly ineffective. Often by the time a patient receives an initial diagnosis, tumor cells are microscopically disseminated throughout the body. The most significant reason for this resistance to therapy is the lack of “total cell kill”. Tumor cells, whether clonogenic or heterogeneous, possess the ability to grow uncontrolled and replace any tumor mass that may be removed. Thus, any effective therapy regimen, whether surgery or drug treatment, must achieve total elimination of the malignant cells. Solid tumors, such as carcinomas, are extremely resistant to most types of therapeutic intervention mainly due to physical inaccessibility of the tumor mass. It is very difficult for a therapeutic agent to reach all of the cells in a solid tumor mass. Surgical intervention may remove the primary tumor however smaller groups of tumor cells, perhaps even microscopic groups, may have already migrated to distant sites in the body where they can re-establish tumor masses. Thus, although surgical intervention and radiation may initially control localized disease, systemic therapy becomes necessary to alleviate metastatic disease. Since many tumors become resistant to standard systemic chemotherapy regimens, development of alternative systemic methods is necessary. The instant invention provides a composition that significantly improves the chances for achievement of total cell kill in the therapy of solid tumors through multi-targeting of disease elements.

In addition to effective systemic treatments, a sensitive method to detect and monitor disease is also essential. The ability to quickly detect cancer inside a patient's body is crucial for accurate diagnosis and successful treatment. Simple and reliable imaging procedures are needed to locate tumors and to monitor the effectiveness of therapy. The use of the multi-targeting composition of the instant invention to image tumors provides advantages as compared with the use of a single targeting composition. Multi-targeting increases sensitivity resulting in a better signal and subsequently a better image. Thus, multi-targeting enables identification of tumors and metastatses at an earlier stage. The tumor imaging approach of the instant invention can be used to determine whether tumor cells are rapidly growing and can help physicians identify whether patients have limited or systemic disease which will better define prognosis and therapeutic options. The increased sensitivity of multi-targeting in imaging allows for a more accurate assessment of the efficacy of treatments.

Extensive research has been conducted in order to improve cancer diagnostics and therapy. Prior artisans have explored a variety of cellular targets or “receptors” in an effort to devise an efficient technique for targeting metastatic disease, while sparing non-diseased tissues. As will be discussed in greater detail in the following sections, these efforts have included targeting of a variety of individual receptors including vascular endothelial growth factor receptor (VEGFR), transferrin receptor and epidermal growth factor receptor (EGFR).

Although each of these receptors have, individually, been shown to have some degree of efficacy in the treatment of cancerous disease, their efficacy is not sufficient to warrant their use as a primary tool in achieving a “cancer-free” status.

The present inventor has devised a unique integrated moiety which does not engender an immunogenic response, and which enables the targeting of multiple receptor sites with one or more imaging or cytotoxic agents, thereby focusing said imaging or cytotoxic agents on a plurality of cell types necessary for tumor growth, viability and metastatic ability. Use of this unique moiety for cancer therapeutics enables a level of reduction in both tumor burden and metastatic development which represents a difference in kind as compared to prior art therapeutics. Additionally, use of this unique moiety for cancer diagnostics enables an increase in sensitivity which represents a difference in kind as compared to prior art diagnostics.

Researchers have attempted to target the tumor vascular supply by making use of vascular endothelial growth factor (VEGF). Several of these attempts are exemplified in the following publications; U.S. Pat. No. 6,451,312 B1 (Thorpe); Wild et al. British Journal of Cancer 83(8):1077-1083 2000; Olson et al. International Journal of Cancer 73(6):865-870 1997; Hotz et al. Journal of Gastrointestinal Surgery 6(2):159-166 2002 and Veenendaal et al. PNAS USA 99(12):7866-7871 2002. In order for a tumor to grow, new blood vessels are required to provide nutrients and oxygen and to remove waste. Tumor cells secrete growth factors to induce the formation of new blood vessels. These newly formed blood vessels are characterized by the expression of surface molecules that are not present on resting endothelium, for example, vascular endothelial growth factor receptor (VEGFR). The VEGFR is internalized into the cell upon binding to its ligand, vascular endothelial growth factor (VEGF). Thus, VEGF can be used as a vector to carry cytotoxic molecules into the non-resting endothelial cells in order to induce a tumor-localized vascular collapse leading to necrosis of tumor cells and subsequently a reduction in the tumor mass.

In addition to targeting receptors specific to the tumor vascular supply, researchers have targeted other cell surface molecules, such as the transferrin receptor which is expressed on both endothelial cells and tumor cells. Transferrin is a vertebrate glycoprotein that functions to bind and transport iron. Uptake of iron is mediated in each individual cell by expression of the transferrin receptor. After binding to iron saturated transferrin the transferrin receptor is internalized to provide the cell with a source of iron. Cells that are actively growing and proliferating show an increased iron requirement, thus these cells also show an increased expression of transferrin receptors. Accordingly, the number of transferrin receptors expressed on the cell surface correlates with cellular proliferation; the highest number expressed on actively growing cells and the lowest number expressed on resting cells. Within the tumor mass, both the tumor cells and the endothelial cells are actively growing and both show an increased expression of transferrin receptors. Various attempts have been made to target transferrin receptors on the cell surface of both tumor and endothelial cells, exemplified in the following patents; U.S. Pat. No. 4,886,780 (Faulk); U.S. Pat. No. 5,000,935 (Faulk); U.S. Pat. No. 5,792,458 (Johnson et al.) and U.S. Pat. No. 5,977,307 (Friden et al.).

Additionally, in their quest to develop more effective systemic therapy, researchers have also attempted to specifically target the tumor cells. It was discovered that since tumor cells exhibit a unique membrane composition as compared to normal cells, the tumor specific molecules can be used as targets for therapy. The epidermal growth factor receptor (EGFR) has been identified as a cell surface receptor that is over-expressed on many types of tumor cells. These receptors (EGFR's) are particularly favorable for targeting purposes since they are internalized into the cell after binding to their ligands (epidermal growth factor, EGF's). Thus, EGF can be utilized as a vector to carry cytotoxic molecules into the interior of tumor cells for enhanced tumor destruction. Many attempts have been made to conjugate various cytotoxic molecules to EGF including, for example, the experiments disclosed in the following publications; Uckun et al. Clinical Cancer Research 4:901-912 1998; Kurihara et al. Cancer Research 59:6159-6163 1999; Yang et al. Journal of Neuro-Oncology 55:19-28 2001 and Lutsenko et al. Tumor Biology 20:218-224 1999.

Although researchers have heretofore constructed systemic therapies aimed at either the tumor cells or the tumor vasculature or have provided combined immunotoxins, they have failed to produce a single non-immunogenic therapeutic moiety capable of effectively targeting multiple disease elements. Since tumors are recognized as comprising a mixed population of cells including both neoplastic cells and normal endothelial cells, what is needed is an efficient composition that is capable of targeting both cellular populations of the tumor mass. What is lacking in the art is a composition comprising a single non-immunogenic compound that can be used to target both the tumor cells and the endothelial cells of the tumor vasculature thereby enabling a multi-targeted approach to cancer diagnosis and treatment.

### DESCRIPTION OF THE PRIOR ART

As is referred to above, prior attempts have been made to target the tumor vascular supply using vascular endothelial growth factor receptor (VEGFR) expression. Representative examples include:

U.S. Pat. No. 6,451,312 B1 (Thorpe) discloses a composition useful for targeting the tumor vasculature comprising VEGF operatively attached to gelonin (protein toxin isolated from the seeds of the plant, Gelonium multiforum). The VEGF of the composition of Thorpe acts as a vector for delivery of gelonin to the interior of endothelial cells. Thorpe suggests combination regimens wherein both the tumor endothelial vasculature and the tumor cells are targeted (see column 4, lines 15-21 and column 14, beginning at line 59 of U.S. Pat. No. 6,451,312 B1). In the method of Thorpe the tumor endothelial vasculature is targeted with an immunological reagent such as an immunotoxin and the tumor cells are targeted with conventional anti-tumor therapy such as radiation or chemotherapy, or through the use of a second immunological reagent. Example II (column 53 of U.S. Pat. No. 6,451,312) exemplifies Thorpe's combination regimen wherein two immunotoxins are used; one targets the tumor endothelial vasculature and one targets the tumor cells. Such immunotoxins are limited in therapeutic efficacy since repeated injections cause a problematic immune response in the host being treated with the immunotoxins. Additionally, Thorpe does not disclose or suggest the use of a single non-immunogenic compound that can be used to target both the tumor cells and the endothelial cells of the tumor vasculature.

Veenendaal et al. (PNAS USA 99(12):7866-7871 2002) also disclose a composition useful for targeting the tumor vasculature comprising VEGF operatively attached to gelonin. The VEGF of the composition of Veenendaal et al. acts as a vector for delivery of gelonin to the interior of endothelial cells.

Wild et al. (British Journal of Cancer 83(8):1077-1083 2000); Olson et al. (International Journal of Cancer 73(6):865-870 1997) and Hotz et al. (Journal of Gastrointestinal Surgery 6(2):159-166 2002) each disclose a composition useful for targeting the tumor vasculature comprising VEGF and diphtheria toxin. The VEGF of these compositions acts as a vector for delivery of diphtheria toxin to the interior of endothelial cells. However, compositions containing diphtheria toxin can not be tolerated over extended periods of time due to the immunogenic reaction produced in the host being treated with the diphtheria toxin.

As is referred to above, prior attempts have been made to target transferrin receptor expression on the cell surface of tumor and endothelial cells. Representative examples include:

Faulk (U.S. Pat. No. 4,886,780) discloses conjugates useful for the treatment of tumors comprising transferrin and anti-tumor drugs. The transferrin of the conjugates of Faulk acts as a vector for delivery of the anti-tumor drugs to the interior of the tumor cells.

Faulk (U.S. Pat. No. 5,000,935) discloses conjugates useful for the imaging and treatment of tumors comprising radiolabled transferrin (¹²⁵I). The transferrin of the conjugates of Faulk acts as a vector for delivery of the radionuclides to the interior of the tumor cells.

Johnson et al. (U.S. Pat. No. 5,792,458) disclose conjugates useful for the treatment of tumors comprising transferrin and mutated diphtheria toxin. The transferrin of the conjugates of Johnson et al. acts as a vector for delivery of diphtheria toxin to the interior of the tumor cells. However, compositions containing diphtheria toxin can not be tolerated over extended periods of time due to the immunogenic reaction produced in the host being treated with the diphtheria toxin.

Friden et al. (U.S. Pat. No. 5,977,307) disclose conjugates useful for the treatment of brain tumors comprising transferrin and a neuropharmaceutical agent such as Nerve Growth Factor (NGF). The transferrin of the conjugates of Friden et al. is targeted to the transferrin receptors expressed on the surface of brain endothelial cells and acts as a vector to deliver the neuropharmacetical agent through the blood-brain barrier to the brain tumor cells. Friden et al. suggest the use of multiple ligands (column 5, lines 40-56 of U.S. Pat. No. 5,977,307) in order to enable the construct to interact more efficiently with the brain capillary endothelial transferrin receptors. In the method of Friden et al. the brain tumor cells are targeted for a therapeutic purpose while the brain capillary endothelial cells are targeted for the purpose of traversal in order for the conjugate to reach the brain tumor cells. Thus, the method of Friden et al. targets only a single disease element (brain tumor cells) as multiple ligands are not used or suggested for the purpose of targeting multiple disease elements.

As is referred to above, prior attempts have been made to target epidermal growth factor receptor (EGFR) overexpression on the surface of tumor cells. Representative examples include:

Uckun et al. (Clinical Cancer Research 4:901-912 1998) disclose a conjugate useful for targeting breast cancer cells comprising EGF and genistein (soybean-derived PTK inhibitor). The EGF of the conjugates of Uckun et al. acts as a vector for delivery of genistein to the interior of breast cancer cells.

Kurihara et al. (Cancer Research 59:6159-6163 1999) disclose a composition useful for targeting brain tumor cells comprising radiolabeled EGF (¹¹¹In) and an anti-transferrin monoclonal antibody (OX26). The EGF of the composition of Kurihara et al. acts as a vector for delivery of radionuclides to the interior of breast cancer cells and the OX26 of the conjugates targets the transferrin receptors expressed on brain capillary endothelium for transfer of the conjugate across the blood-brain barrier. In the method of Kurihara et al. the brain tumor cells are targeted for a therapeutic purpose while the brain capillary endothelial cells are targeted only for the purpose of traversal of the blood-brain barrier in order for the conjugate to reach the brain tumor cells. Thus, the method of Kurihara et al. targets only a single disease element (brain tumor cells) as the transferrin receptor is not targeted as a disease element.

Yang et al. (Journal of Neuro-Oncology 55:19-28 2001) disclose a composition useful for targeting brain tumor cells comprising radiolabeled EGF (^{99m}Tc). The EGF of the composition of Yang et al. acts as a vector for delivery of radionuclides to the interior of breast cancer cells.

Lutsenko et al. (Tumor Biology 20:218-224 1999) disclose compositions useful for targeting breast cancer cells and melanoma cells comprising EGF and phthalocyanines. The EGF of the composition of Lutsenko et al. acts as a vector for delivery of phthalocyanines to the interior of breast cancer cells and melanoma cells.

An important distinction between the instant invention and the prior art involves the source of experimental tumors. Tumors grown in immunodeficient mice which are derived from cell lines often develop vasculature of murine origin. The VEGF isoform used in the instant invention is of human origin and thus will react only with VEGF receptors on endothelial cells of human origin. The compositions of the instant invention containing VEGF would be ineffective if used against murine blood vessels. The tumors targeted in the experiments described herein are all derived from human surgical specimens and exhibit vasculature of human origin (see FIG. 4). In contrast, the tumors which are targeted in the experiments disclosed in the above-referenced prior art are all derived from cell lines and hence would exhibit blood vessels of murine origin. Thus, the instant invention provides an improved model system for targeting angiogenesis in human tumors.

Another important distinction between the instant invention and the prior art is that the composition described in the instant invention contains only human proteins which will be non-immunogenic when administered to a human patient. This is in contrast to prior art treatments which utilize immunotoxins and bacterial toxins which produce immune reactions when administered to a human patient.

Additionally, it is important to note that in contrast to the instant invention, none of the above references discuss or suggest a composition comprising a single non-immunogenic compound that can be used to target both the tumor cells and the endothelial cells of the tumor vasculature thereby enabling a multi-targeted approach to cancer diagnosis and treatment.

### SUMMARY OF THE INVENTION

The instant invention provides a composition comprising a single non-immunogenic compound that can be used to target both the tumor cells and the endothelial cells of the tumor vasculature thereby enabling a multi-targeted approach to cancer diagnosis and treatment. The composition of the instant invention can function both as a visualizing agent and as a therapeutic agent. The multi-targeting ability of the composition allows for increased sensitivity for imaging and increased efficacy for the reduction of tumor burden when compared with compositions available in the prior art.

The composition of the instant invention contains human vascular endothelial growth factor (VEGF) and human epidermal growth factor (EGF) each operatively linked to radiolabeled human transferrin, wherein said human VEGF binds to human VEGF receptors on endothelial cell surfaces of intratumoral blood vessels, said human EGF binds to human EGF receptors when present on cell surfaces of tumor cells and said radiolabeled human transferrin binds human transferrin receptors on endothelial cell surfaces of intratumoral blood vessels and cell surfaces of tumor cells. FIG. 1 shows a schematic diagram of the compositions described herein.

As used herein, the term vascular endothelial growth factor (VEGF) encompasses VEGF and isolated peptide fragments or biologically active portions thereof, analogues of VEGF and any biologically active portion thereof and any molecules and portions of molecules having the biological activity of VEGF.

As used herein, the term transferrin encompasses transferrin and isolated peptide fragments or biologically active portions thereof, analogues of transferrin and any biologically active portion thereof and any molecules and portions of molecules having the biological activity of transferrin.

As used herein, the term epidermal growth factor (EGF) encompasses EGF and isolated peptide fragments or biologically active portions thereof, analogues of EGF and any biologically active portion thereof and any molecules and portions of molecules having the biological activity of EGF.

As used herein, the term “bioactivity” refers to the ability of a ligand to bind to its complementary receptor thus enabling internalization of the ligand into the cellular interior.

As used herein, the term “biologically active portion” refers to the portion of a ligand that has the ability to bind to its complementary receptor thus enabling internalization of the ligand into the cellular interior.

As used herein, the phrase “host suspected of having a tumor” refers to a host wherein the presence of a tumor is not confirmed. The composition of the instant invention is administered once to a host suspected of having a tumor for diagnostic purposes (imaging).

As used herein, the phrase “host having a tumor” refers to a host wherein the presence of a tumor has been confirmed. The composition of the instant invention is administered sequentially to a host having a tumor for therapeutic purposes.

With regard to the composition of the instant invention, vascular endothelial growth factor (VEGF) acts as a vector for delivery of radiolabeled transferrin to the endothelial cells of the tumor vasculature and epidermal growth factor (EGF) acts as a vector for delivery of radiolabeled transferrin to the tumor cells. Transferrin acts as a dual-functioning vector for delivery of radionuclides to both the tumor cells and the endothelial cells of the tumor vasculature.

The radionuclides are bound in the iron-binding sites of the transferrin molecule. These radionuclides can function as either imaging agents or as cytotoxic agents. The imaging approach can be used as a therapeutic approach based on the number of sequential doses administered to a host. The amount of radiation applied to the composition for both imaging and treatment is identical, however one dose is administered for the purpose of imaging and multiple doses are administered over a period of time for the purpose of treatment. The period of time between doses is selected based upon the needs of the host receiving the treatment. Illustrative, albeit non-limiting examples of periods of time allowed between doses are hours, days and weeks. A particularly preferred period of time between doses is one week, the use of which is illustrated in the examples herein. A therapeutic dose is administered each selected period of time until a statistically significant inhibition of tumor growth is achieved. The amount of inhibition is determined by comparison of tumor growth in treated animals with tumor growth in control animals which have not received treatments. The bar graphs shown in FIGS. 5 and 7 demonstrate results achieved after five weeks of therapy. Illustrative, albeit non-limiting examples of radionuclides known and commonly used in the art for radioactive labeling are ¹²³I, ¹²⁵I, ¹³⁰I, ¹³¹I , ¹³³I , ¹³⁵I , ⁴⁷Sc , ⁷²As , ⁷²Se, ⁹⁰Y, ⁸⁸Y, ⁹⁷Ru, ¹⁰⁰Pd, ^{101m}Rh, ¹¹⁹Sb, ¹²⁸Ba, ¹⁹⁷Hg , ²¹¹At , ²¹²Bi, ¹⁵³Sm, ¹⁶⁹Eu, ²¹²Pb, ¹⁰⁹Pd, ¹¹¹In, ⁶⁷Ga ⁶⁸Ga , ⁶⁷Cu, ⁷⁵Br, ⁷⁶Br, ⁷⁷Br, ^{99m}Tc, ¹¹C, ¹³N, ¹⁵O and ¹⁸F. A particularly preferred radiolabel is ¹¹¹In, the use of which is illustrated in the examples herein.

The composition of the instant invention can be added to a pharmacologically effective amount of a carrier to provide a pharmaceutical composition for administration to an animal host, including administration to a human patient. Illustrative, albeit non-limiting examples of carriers known in the art and suitable for use with the instant invention are water, saline solutions and dextrose solutions. A particularly preferred carrier is saline, the use of which is illustrated in the examples herein.

Accordingly, it is an objective of the instant invention to provide a compound comprising human vascular endothelial growth factor (VEGF)and human epidermal growth factor (EGF) operatively linked to radiolabeled human transferrin, wherein human VEGF binds human VEGF receptors on the endothelial cell surface of intratumoral blood vessels, human transferrin binds human transferrin receptors on endothelial cell surfaces of intratumoral blood vessels and cell surfaces of tumor cells and wherein human EGF binds human EGF receptors when present on the cell surface of tumor cells.

It is another objective of the instant invention to provide a pharmaceutical composition comprising the compound of the instant invention and a pharmacologically effective amount of a carrier.

It is a further objective of the instant invention to provide a conjugate consisting essentially of human vascular endothelial growth factor (VEGF)and human epidermal growth factor (EGF) operatively linked to radiolabeled human transferrin, wherein human VEGF binds human VEGF receptors on the endothelial cell surface of intratumoral blood vessels, human transferrin binds human transferrin receptors on endothelial cell surfaces of intratumoral blood vessels and cell surfaces of tumor cells and wherein human EGF binds human EGF receptors when present on the cell surface of tumor cells.

It is yet another objective of the instant invention to provide a pharmaceutical composition comprising the conjugate of the instant invention and a pharmacologically effective amount of a carrier.

Other objectives and advantages of this invention will become apparent from the following description taken in conjunction with the accompanying drawings wherein are set forth, by way of illustration and example, certain embodiments of this invention. The drawings constitute a part of this specification and include exemplary embodiments of the present invention and illustrate various objects and features thereof.

### BRIEF DESCRIPTION OF THE FIGURES

FIG. 1 shows a diagrammatic presentation of the compositions described herein.

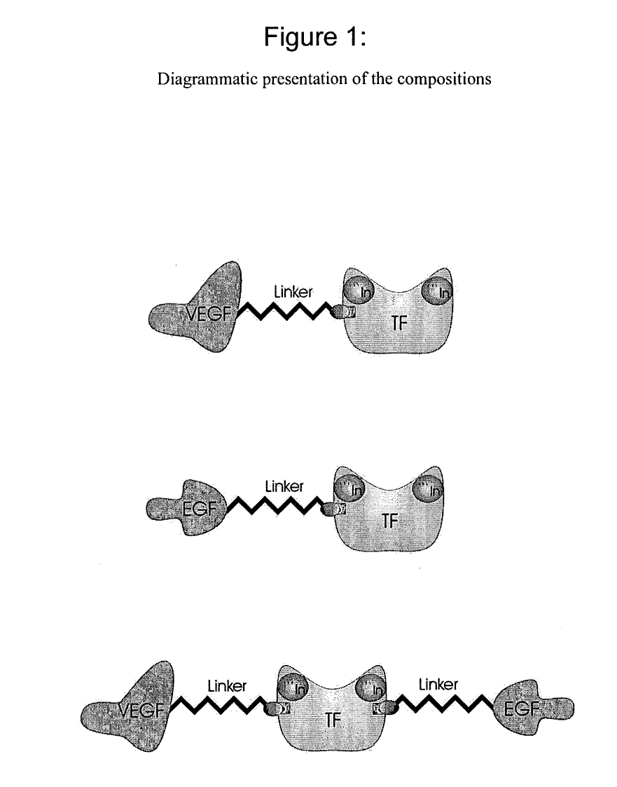

FIG. 2 shows a graphical presentation of Breast Cancer Bone Metastatsis (BCBM) volumes in SCID mice.

FIGS. 3A-3B show immunohistochemistry of BCBM specific for EGFR (epidermal growth factor receptor). FIG. 3A shows a histologic section stained with antibody (TS40) specific for the human cell surface EGFR. FIG. 3B is a micrograph showing an isolated EGFR⁺ breast cancer cell in the bone marrow.

FIG. 4 is a micrograph showing blood vessels of human origin in the BCBM tumors in SCID mice.

FIG. 5 shows a graphical presentation of the inhibition of breast cancer growth achieved by treatment with the composition of the instant invention.

FIG. 6 shows imaging test results in mice.

FIG. 7 shows a graphical presentation comparing inhibition of breast cancer growth achieved by treatment with various compositions including the composition of the instant invention.

### DEFINITIONS

The following list defines terms, phrases and abbreviations used throughout the instant specification. Although the terms, phrases and abbreviations are listed in the singular tense the definitions are intended to encompass all grammatical forms.

As used herein, the abbreviation “EGF” refers to epidermal growth factor.

As used herein, the abbreviation “EGFR” refers to epidermal growth factor receptor.

As used herein, the abbreviation “VEGF” refers to vascular endothelial growth factor.

As used herein, the abbreviation “VEGFR” refers to vascular endothelial growth factor receptor.

As used herein, the abbreviation “BCBM” refers to breast cancer bone metastatsis.

As used herein, the abbreviation “PEG” refers to polyethylene glygol.

As used herein, the abbreviation “TF” refers to transferrin.

As used herein, the abbreviation “SA” refers to streptavidin.

As used herein, the abbreviation “TF/SA” refers to a compound comprising transferrin linked to streptavidin.

As used herein, the abbreviation “MBS” refers to m-maleimidobenzoyl N-hydroxysuccinimide ester.

As used herein, the abbreviation “HPLC” refers to high performance liquid chromatography.

As used herein, the abbreviation “RP-HPLC” refers to reverse phase high performance liquid chromatography.

As used herein, the abbreviation “NHS” refers to N-hydroxysuccinimide.

As used herein, the abbreviation “TFA” refers to trifluoroacetic acid.

As used herein, the abbreviation “PBS” refers to phosphate buffered saline.

As used herein, the abbreviation “SCID” refers to a type of transgenic mouse that is severe combined immuno-deficient.

As used herein, the term “selective delivery” is defined as delivery which is targeted to a specific cell type for the purpose of avoiding uniform or even delivery to all cell types.

As used herein, the term “ligand” refers to a molecule that exhibits specific binding of high affinity for another molecule and upon binding with that molecule is internalized into the cellular interior. An illustrative, albeit non-limiting example of how the term “ligand” is used in the context of the instant specification is a protein ligand binding to a cell surface receptor, such as EGF binding to the EGFR.

As used herein, the term “receptor” refers to a molecule that exhibits specific binding of high affinity for its complementary ligand. An illustrative, albeit non-limiting example of how the term “receptor” is used in the context of the instant specification is a cell surface receptor binding to a ligand, such as the EGFR binding the EGF.

As used herein, the term “complementary receptor” refers to the receptor a ligand specifically binds with high affinity, for example, the EGFR is the complementary receptor for EGF.

As used herein, the term “target” refers to a specific molecule expressed on the cellular surface such as a receptor to which a specific moiety can be directed, for example the EGFR is a target for EGF.

As used herein, the term “targeting agent” refers to a specific molecule that binds to a complementary molecule expressed on the cellular surface such as a ligand, for example EGF is a targeting agent for the EGFR.

As used herein, the phrase “multi-targeted” refers to the ability of a therapeutic protocol to target at least two disease elements, for example, the composition of the instant invention can be used to target an entire tumor mass by using EGF to target the tumor cells, by using VEGF to target the endothelial cells of the tumor vasculature and by using transferrin to target both the tumor cells and the endothelial cells of the tumor vasculature.

As used herein, the phrase “disease elements” refers to the separate targets or elements that contribute to result in an entire disease state, for example, malignant cells and endothelial cells are each separate disease elements in cancer pathology.

As used herein, the term “VEGF” refers to a glycosylated polypeptide that serves as a mitogen to stimulate vascular development. VEGF imparts activity by binding to vascular endothelial cell plasma membrane-spanning tyrosine kinase receptors (VEGFR's) which then activates signal transduction.

As used herein, the term “VEGFR” refers to a vascular endothelial cell plasma membrane-spanning tyrosine kinase receptor which binds VEGF thus exerting a mitogenic signal to stimulate vascularization of tissues.

As used herein, the term vascular endothelial growth factor (VEGF) encompasses VEGF and isolated peptide fragments or biologically active portions thereof, analogues of VEGF and any biologically active portion thereof and any molecules and portions of molecules having the biological activity of VEGF.

As used herein, the term “EGF” refers to a mitogenic polypeptide that exhibits growth stimulatory effects for epidermal and epithelial cells. EGF imparts activity by binding to epidermal and/or epithelial cell plasma membrane-spanning tyrosine kinase receptors (EGFR's) which then activates signal transduction.

As used herein, the term “EGFR” refers to a epidermal and/or epithelial cell plasma membrane-spanning tyrosine kinase receptor which binds EGF thus exerting a mitogenic signal.

As used herein, the term epidermal growth factor (EGF) encompasses EGF and isolated peptide fragments or biologically active portions thereof, analogues of EGF and any biologically active portion thereof and any molecules and portions of molecules having the biological activity of EGF.

As used herein, the term “transferrin” refers to a vertebrate glycoprotein that functions to bind and transport iron.

As used herein, the term “transferrin receptor” refers to a receptor expressed on the surface of cells functioning to capture and bind iron saturated transferrin. Expression of the transferrin receptor is increased in cells which are actively proliferating.

As used herein, the term transferrin encompasses transferrin and isolated peptide fragments or biologically active portions thereof, analogues of transferrin and any biologically active portion thereof and any molecules and portions of molecules having the biological activity of transferrin.

As used herein, the term “host” refers to any animal having a tumor, including a human patient.

As used herein, the term “tumor tissue” refers to all of the cellular types which contribute to formation of a tumor mass, including tumor cells and endothelial cells, for example, the tumor tissue includes tumor cells and blood vessels.

As used herein, the term “tumor mass” refers to a foci of tumor tissue.

As used herein, the term “inhibition” refers to retarding the growth of a tumor mass.

As used herein, the term “bioactivity” refers to the ability of a ligand to bind to its complementary receptor thus enabling internalization of the ligand into the cellular interior.

As used herein, the term “biologically active portion” refers to the portion of a ligand that has the ability to bind to its complementary receptor thus enabling internalization of the ligand into the cellular interior.

As used herein, the phrases, “tumor vasculature”, “tumor endothelium” and “tumor vessels” all refer to the circulatory vessels which supply the tumor tissue with blood.

As used herein, the term “angiogenesis” refers to the process by which tissues become vascularized. Angiogenesis involves the proteolytic degradation of the basement membrane on which the endothelial cells reside followed by the chemotactic migration and mitosis of the endothelial cells to support a new capillary shoot.

As used herein, the term “linker” refers to the molecules which join the ligands of the composition of the instant invention together to form a single compound; for example, EGF-PEG attached to biotin links streptavidin attached to transferrin.

As used herein, the phrase “operatively linked” means that the linkage does not destroy the functions of each of the separate elements of the composition of the instant invention, for example, when linked together by a linker to form the single composition of the instant invention the ligands retain the ability to bind their complementary receptors.

As used herein, the term “carrier” refers to a pharmaceutically inert substance that facilitates delivery of an active agent to a host, for example, as is shown in the experiments described herein, saline functions as a carrier for delivery of the composition of the instant invention to the mouse host.

As used herein, the phrase “pharmacologically effective amount of a carrier” refers to an amount of a carrier that is sufficient to effectively deliver an active agent to a host.

As used herein, the term “pharmaceutical composition” refers to the compositions of the instant invention combined with a pharmacologically effective amount of a carrier.

The phrases “tumor endothelium”, “tumor vessels” and “tumor vasculature” are used interchangeably herein.

The terms “tumor cell”, “neoplastic cell” and “cancer cell” are used interchangeably herein.

As used herein, the term “compound” refers to a substance containing at least two distinct elements to which an unlimited number of other elements can be added.

As used herein, the term “conjugate” refers to a substance containing at least two distinct elements and a defined number of additional elements.

As used herein, the term “composition” is intended to encompass both a compound and a conjugate.

### DETAILED DESCRIPTION OF THE INVENTION

Experimental Procedures

Sequences

The following nucleic acid sequences and corresponding amino acid sequences were used to generate the DNA and polypeptides used in the experiments described herein. Homo sapiens (human) VEGF165 (vascular endothelial growth factor isoform 165) nucleic acid sequence is disclosed as SEQ ID NO:1 and translates into VEGF165 protein disclosed as amino acid sequence SEQ ID NO:2. Homo sapiens (human) transferrin nucleic acid sequence is disclosed as SEQ ID NO:3 and translates into transferrin protein disclosed as amino acid sequence SEQ ID NO:4. Homo sapiens (human) EGF (epidermal growth factor) nucleic acid sequence is disclosed as SEQ ID NO:5 and translates into EGF protein disclosed as amino acid sequence SEQ ID NO:6.

Linkers

When assembling compositions from multiple elements, elements are either linked directly through chemical conjugation (for example through reaction with an amine or sulfhydryl group) or are linked indirectly through molecules termed linkers. When selecting a linker it is important to choose the appropriate length and flexibility of linker in order to reduce steric hindrance between the elements of the composition. For example, if an element of a composition is brought into close physical proximity of another element by linkage, the function of either or both elements can be affected. Each element of the composition must retain its bioactivity, for example in the instant invention, each ligand must retain its ability to bind to its complementary receptor after linkage with the other two ligands of the composition. Illustrative, albeit non-limiting examples of linkers are glycols, alcohols and peptides. Particularly preferred linkers are PEG (polyethylene glycol) and the peptide linker shown as SEQ ID NO:8 (use of each of these linkers is illustrated in the examples described herein).

Crosslinking of VEGF (and EGF) to a Biotinylated-polylinker

EGF and VEGF are crosslinked to a biotinylated polylinker by carrying out the following protocol. The polylinker used consists of 15 amino acid residues shown as SEQ ID NO:8. The cDNA sequence encoding this polylinker is shown as SEQ ID NO:7. The first glycine residue at the N-terminal was biotinylated. EDC (1-Ethyl-3-(3-Dimethylaminopropyl)carbodiimide Hydrochloride) and NHS (N-Hydroxysuccinimide) were equilibrated to room temperature. 0.4 mg of EDC and 0.6 mg of NHS were added to 1 mg/ml of the polylinker peptide solution (in activation buffer: 0.1 M MES (2-[N-morpholino]ethane sulfonic acid), 0.5 M NaCl, pH 6.0) to a final concentration of EDC and NHS of 2 mM and 5 mM respectively. The reaction mixture was then held for 15 minutes at room temperature. 1.4 ul of 2-mercaptoethanol was then added (to a final concentration of 20 mM). The reaction mixture was then run through P2 gel filtration mini-column and eluted by the activation buffer. Fractions containing the protein were then pooled together. Equal mole:mole ratios of either VEGF or EGF protein were added to the pooled fractions and reacted for 2 hours at room temperature. Hydroxylamine was added to a final concentration of 10 mM and the VEGF-linker or EGF-linker was purified by P2 gel filtration mini-column.

Synthesis of TF/SA Compound

8.84 mg of transferrin (TF) was thiolated by adding a 5-fold molar excess of 2-Iminothiolane hydrochloride (Traut's reagent) in pH 8.0, 0.16 M borate. Following 90 minutes at room temperature, the thiolated TF was desalted and concentrated by Centricon microconcentrators. Ellman's reagent (Pierce) was then used to demonstrate that a single thiol group was inserted on the surface of TF. 7 mg of streptavidin (SA) (in PBS) was activated by adding to a 20:1 molar ratio of m-maleimidobenzoyl N-hydroxysuccinimide ester (MBS)(stock at 1 mg/ml in dimethylformamide). After 20 minutes, the activated SA was desalted on a microconcentrator and immediately, the activated SA was added to a 10 molar excess of thiolated TF. They were mixed and then incubated at room temperature for 3 hours. Purification of the TF/SA compound was done by HPLC using TSK-G3000 column. The number of biotin binding sites per TF/SA compound was determined with ³H-biotin binding assay.

Conjugation of VEGF-linker-biotin (and EGF-linker-biotin) to TF-SA and ¹¹¹In-labeling

VEGF-Linker-Biotin and EGF-Linker-Biotin are each added to TF/SA by carrying out the following protocol. The compound of VEGF-Linker-biotin and EGF-Linker-biotin and TF/SA was prepared by mixing 5 nmol of VEGF-Linker-biotin (or 5 nmol of EGF-Linker-biotin) with 8 nmol of TF/SA (1:1.6 molar ratio). HPLC was then used to purify the VEGF-Linker-biotin-TF-SA-EGF-Linker-biotin compound. The reaction mixture was then applied to a TSK-gel G3000 SW_{XL} HPLC gel filtration column, followed by elution in 0.01 M Na₂HPO₄/0.15 M NaCl/pH 7.4/0.05% Tween-20 at a flow rate of 0.5 mL/min for 40 minutes, and 0.5 mL fractions were collected. 2 mCi ¹¹¹In acetate was mixed with the compound in 10 mM HEPES, 15 mM NaHCO3 pH 7.4 buffer for 1 hour at room temperature. Free ¹¹¹In was separated from bound ones by running the reaction volume through P2 (BioRad) size-exclusion chromatography using a mini-column and the ¹¹¹In bound-protein was eluted with pH 7.4 10 mM HEPES, 15 mM NaHCO3 buffer. Fractions collected (100 μl) were measured for radioactivity and fractions containing the protein were combined and the specific radioactivity of proteins was determined. ¹¹¹In-labeled proteins were used immediately.

Conjugation of VEGF (and EGF) to PEG3400-biotin

Alternatively to linkage with a peptide linker, VEGF and EGF can also be linked to transferrin using PEG by carrying out the following protocol. NHS-PEG3400-biotin was obtained from Shearwater Polymers (Huntsville, Ala.), where NHS=N-hydroxysuccinimide and PEG3400=poly(ethylene glycol) of 3400 Da molecular mass. NHS-PEG3400-biotin (20 nmol in 310 μl of 0.05 M NaHCO3) was added in a 1:1 molar ratio to either VEGF or EGF (16 nmol in 250 μl of 0.05 M NaHCO3) followed by incubation at room temperature for 60 minutes. The mixture was then applied to two Sepharose 12 HR 10/30 FPLC columns in series, followed by the elution in 0.01 M NaH2PO4/0.15 M NaCl/pH 7.5 at a flow rate of 0.7 mL/minute for 120 minutes. Fraction(s) that contained VEGF or EGF bound to PEG3400-biotin moiety were pooled together.

Conjugation of VEGF-PEG3400-biotin (and EGF-PEG3400-biotin) to TF-SA and ¹¹¹In-labeling

Following reaction of EGF and/or VEGF with NHS-PEG3400-biotin and transferrin with streptavidin, both compounds were purified by HPLC. The EGF (and/or VEGF)-NHS-PEG3400-biotin and TF/SA compounds were then mixed (1:1.6 molar ratio). The compounds EGF (and/or VEGF)-NHS-PEG3400-biotin-TF-SA was purified by HPLC and labeled with ¹¹¹In by mixing with ¹¹¹In acetate and purified on a P-2 size-exclusion mini-column. The specific activity of ¹¹¹In-EGF (and/or VEGF)-PEG3400-biotin-TF-SA compounds were about 100-400 mCi/mg.

Experimental Mice

Severe combined immuno-deficient C.B.-17 scid/scid (SCID) mice were bred and maintained according to the protocol of Sandhu et al. (Critical Reviews in Biotechnology 16(1):95-118 1996). Mice were used when 6-8 weeks old and were pre-treated with a dose of 3 Gy γ-radiation administered from a ¹³⁷CS source (Gamacell, Atomic Energy of Canada Ltd. Commercial Products). The irradiated SCID mice receive intraperitoneal injection of 20 μl ASGM1 sera diluted to 100 μl with saline, 4 hours pre-bone transplantation and every 7 days thereafter for the duration of the experiments.

Experimental Tumors

The composition of the instant invention is effective when used to target either an EGFR⁺ tumor or an EGFR⁻ tumor since the transferrin moiety targets those tumor cells that are EGFR⁻. A bone metastatic focus of a primary EGFR⁺ breast tumor was used in the experimental examples herein described. However, it is noted that the use of the composition of the instant invention in breast tumors is an illustrative example only and is not intended to limit the use of the composition to breast tumors. The composition of the instant invention can be administered to a host having any tumor comprising cells which are positive for the expression of at least one of the cell surface receptors described herein (the transferrin receptor, the EGFR and the VEGFR).

Implantation of Human Breast Cancer Bone Metastasis in Scid Mice

Breast cancer bone metastasis (BCBM) specimens (n=20, JJ1 to JJ20) were obtained from female patients (age range 40-68 years) undergoing total hip joint replacement due to BCBM mediated bone osteolysis. The majority (70%) of the BCBM used in these experiments were infiltrative ductal carcinoma and each specimen was assigned a number JJ1 to JJ20. Normal cancellous bone was obtained from healthy adult patients (age range 59-80 years) undergoing total hip joint replacement for the treatment of degenerative osteoarthritis. The BCBM was obtained from the proximal femur, morcellized using a rongeur and maintained under sterile conditions in RPMI (1640) medium (Gibco BRL, Burlington Ont. Canada). Transplantation of the normal bone and BCBM into mice was performed within 2 hours of procurement, under a general anesthetic (intramuscular administration of Xylazine (4 μl/20 g mouse), and Ketamine (4 μl/20 g mouse) in 40 μl of 0.9% sodium chloride) under sterile conditions. Morcellized normal bone (Bone-SCID mice), and BCBM (BCBM-SCID mice), approximately 0.121 cm³ per mouse, was transplanted subcutaneously over the left flank in SCID mice (n=30).

Tumor Measurement

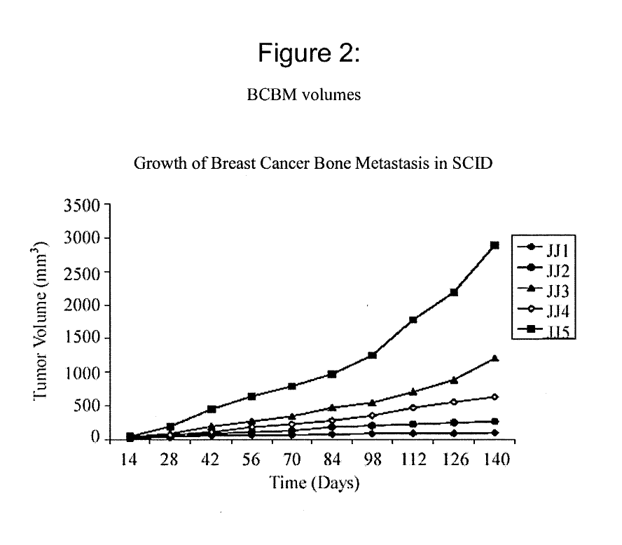

BCBM volumes were measured every 14 days for 20 weeks to assess tumor growth in SCID mice as described by Osborne et al. (Cancer Research 45:584-590 1985). The data shows that in contrast to the similar growth rate of breast cancer cell lines in immunodeficient mice the growth pattern of BCBM specimens varies in SCID mice (see FIG. 2). Results showed JJ5 gave the best growth of the tumor, thus it was chosen as the surgical specimen for use in subsequent in vitro cell studies and in vivo animal experiments.

Cell Culture Studies

Measurement of EGF-¹¹¹In-labeled Transferrin Compound Binding to Breast Cancer Cells

Breast cancer cells express up to 100-fold higher levels of EGFR than do normal epithelial tissues. EGFR expression in breast cancer bone metastasis biopsies ranged from 1-1300 fmol/mg membrane protein (approximately 400-1,000,000 receptors/cell) and was associated with high relapse rate and poor long term survival. Normal epithelial cells express <10⁴ receptors/cell.

For the normal breast cell line HBL-100, 8000 EGFR/cell has been reported. The expression of EGFR in breast cancer cell lines has a reported range of 800 EGFR/cell for MCF-7 cells to 10⁶ EGFR/cell for MDA-MB-468 cells. The liver is the only normal tissue exhibiting moderate levels of EGFR (8×10⁴ to 3×10⁵ receptors/cell) likely reflecting its role in the elimination of EGF from the blood. Utilizing the Auger electron emitter ¹¹¹In was used in the initial experiments to illustrate the utility of the invention using EGF-¹¹¹In-labeled transferrin compounds. The EGF-¹¹¹In-labeled transferrin (0.25-80 ng) was incubated with 1.5×10⁶ cells/dish JJ5 Breast Cancer (prepared from BCBM JJ5) cells in 1 mL of 0.1% human serum albumin in 35 mm multiwell culture dishes at 37° C. for 30 minutes. The cells were transferred to a centrifuge tube and centrifuged. The cell pellet was separated from the supernatant and counted in a g-scintillation counter to determine bound (B) and free (F) radioactivity. Non-specific binding was determined by conducting the assay in 100 nM hEGF. The kinetics of binding was determined by incubating 1 ng of EGF-¹¹¹In-labeled transferrin compound with 3×10⁶ JJ5 Breast Cancer cells at 37° C. and determining the proportion of radioactivity bound to the cells at various times up to 24 hours. Internalized fraction was measured by determining the proportion of radioactivity which could not be displaced from the cell surface by 100 nM hEGF. Cell-associated binding (surface-binding and intracellular accumulation) was expressed as a percentage of medium radioactivity bound per mg of cell study protein.

The affinity constant for binding of EGF-¹¹¹In-labeled transferrin compound to JJ5 cells was 8×10⁸ L/mol and the number of binding sites was 2.7×10⁶. EGF-¹¹¹In-labeled transferrin compound was rapidly bound by the breast cancer cells and retained for at least 24 hours. Over a 24 hour period at 37° C., <8% was lost from the cells in vitro.

The Growth Inhibition Assay of EGF-¹¹¹In-labeled Transferrin Compound against JJ5 Breast Cancer Cells

JJ5 breast cancer cells (prepared from BCBM JJ5) expressing approximately 10⁶ epidermal growth factor receptors/cell were incubated with EGF-¹¹¹In-labeled transferrin compound, unlabeled hEGF or ¹¹¹In-oxine, centrifuged to remove free ligand, then assayed and seeded (10⁶ cells/dish) into 35 mm culture dishes. Growth medium was added and the cells were cultured at 37° C./5% CO² for 4 days. The cells were then recovered by trypsinization and counted in a hemocytometer. Control dishes contained cells cultured in growth medium containing ¹¹¹In-DTPA or growth medium alone.

The growth inhibition assay of EGF-¹¹¹In-labeled transferrin compound (3.4 pCi/cell) achieved a 83% growth inhibition of the JJ5 cells compared to the medium control, whereas ¹¹¹In oxine (3.5 pCi/cell) which enters all the cells resulted in 91% growth inhibition.

Cytotoxicity Assay of EGF-¹¹¹In-labeled Transferrin Compound against JJ5 Breast Cancer Cells

JJ5 breast cancer cells were incubated with increasing amounts EGF-¹¹¹In-labeled transferrin compound or ¹¹¹In-oxine, centrifuged to remove free ligand, assayed and then seeded into 50 mm culture dishes. The number of cells seeded was varied from 3×10⁴ to 3×10⁶cells to obtain approximately 400 viable colonies/dish taking into account the plating efficiency and the expected level of cytotoxicity. Control dishes contained JJ5 breast cancer cells which were incubated with normal saline. Growth medium was added and the cells were cultured at 37° C./5% CO² for 14 days. The growth medium was removed and the colonies were stained with methylene blue (1% in a 1:1 mixture of ethanol and water) then washed twice. The number of colonies per dish was counted using a manual colony counter (Manostat Corp). The plating efficiency was calculated by dividing the number of colonies observed by the number of cells seeded in each dish. The surviving fraction at increasing amounts of EGF-¹¹¹In-labeled transferrin compound or ¹¹¹In-oxine was calculated by dividing the plating efficiency for dishes containing treated cells with that observed for control dishes with normal saline.

Using a colony-forming assay, the radiotoxicity of internalization for JJ5 breast cancer cells was evaluated. EGF-¹¹¹In-labeled transferrin compounds (8 pCi/cell) resulted in a 99% reduction in cell survival for JJ5 cells. ¹¹¹In-oxine was also radiotoxic with greater than 99% cell killing at <6 pCi/cell.

There are various advantages of using the radiolabeled compositions of the instant invention in cancer therapy. As seen from the foregoing data, EGF-¹¹¹In-labeled transferrin compositions are rapidly internalized by cancer cells. The internalization process for EGF-¹¹¹In-labeled transferrin compositions involves an active transport mechanism utilizing the EGFR binding domain of the compound, rather than simple diffusion across the cell membrane. This active transport mechanism for the composition probably also includes nuclear translocation, as for the case of EGF, which allows for a maximal radiation dose of Auger electrons to be delivered to the cell's DNA. The compositions of the instant invention employ human polypeptides and are not immunogenic in humans. EGF-¹¹¹In-labeled transferrin compositions have been shown to retain ¹¹¹In over a 24 hour period at 37° C., with <8% of ¹¹¹In radioactivity was lost from cells in vitro. These characteristics are important for cell killing.

Immunohistochemistry Staining and Measurement of EGF Receptor on BCBM Cells

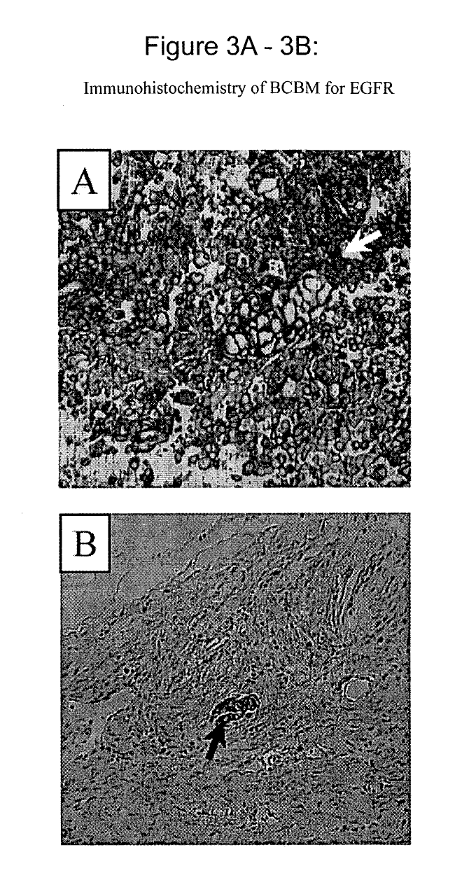

Immunohistochemistry of BCBM pre-implanted into mice showed all the specimens (n=20) had breast cancer cells negative for the estrogen and progesterone receptors (data not shown). Normal human bone histological sections were used as controls, no staining was observed in these specimens (data not shown). BCBM were retrieved from the mice at 20 weeks. Histologic sections were fixed and prepared. Immunohistochemical staining was done using mouse anti-EGF-receptor monoclonal antibody (TS40). In contrast to the implants and the controls, 16 of the 20 BCBM specimens had breast cancer cells positive for human EGFR (see FIGS. 3A-B). The white arrow in FIG. 3A points out a dense mass of EGFR⁺ cells. The arrow in FIG. 3B points out an isolated EGFR⁺ cell in the bone marrow. Mean (±SDEV) expression levels of EGF receptor was measured on breast cancer cells from tumor JJ5 by radioligand binding assay 24 and were in the range of 2.7 (±0.8)×10⁶ receptors/cell.

Immunohistochemistry Staining of BCBM Human Blood Vessels

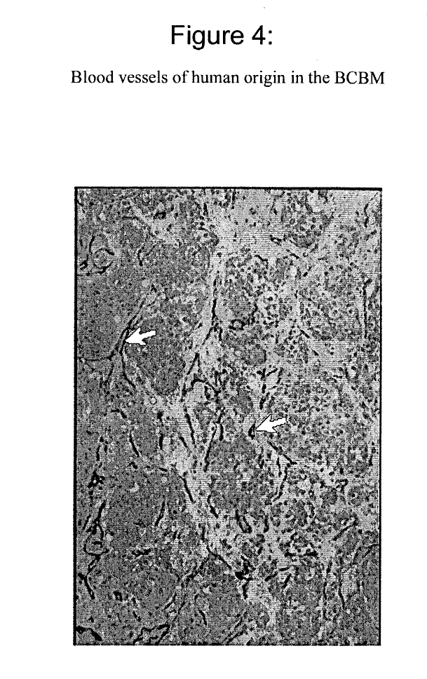

To evaluate the role of angiogenesis in the growth of human breast carcinoma, human BCBM surgical specimens were implanted in SCID mice. The breast tumors showed numerous blood vessels infiltrating the central mass of the tumors. In order to accurately assess the efficacy of treatment using the composition of the instant invention against human tumors, the blood vessels which developed in the BCBM in the mice must be of human origin. Immunohistochemical staining was done on BCBM sections using mouse anti-human CD34 antibody. Anti-human CD34 reacts specifically with human blood vessels and thus will not react with murine blood vessels. As shown in FIG. 4, these results clearly demonstrate the presence of human blood vessel angiogenesis within the tumor xenografts retrieved from SCID mice at 20 weeks. In FIG. 4, the arrow points out the dark blood vessels of human origin (stained with anti-human CD34), thus these specimens can be used to accurately assess the efficacy of the VEGF portion of the composition of the instant invention.

Animal Studies

Effect of Compositions on BCBM Growth

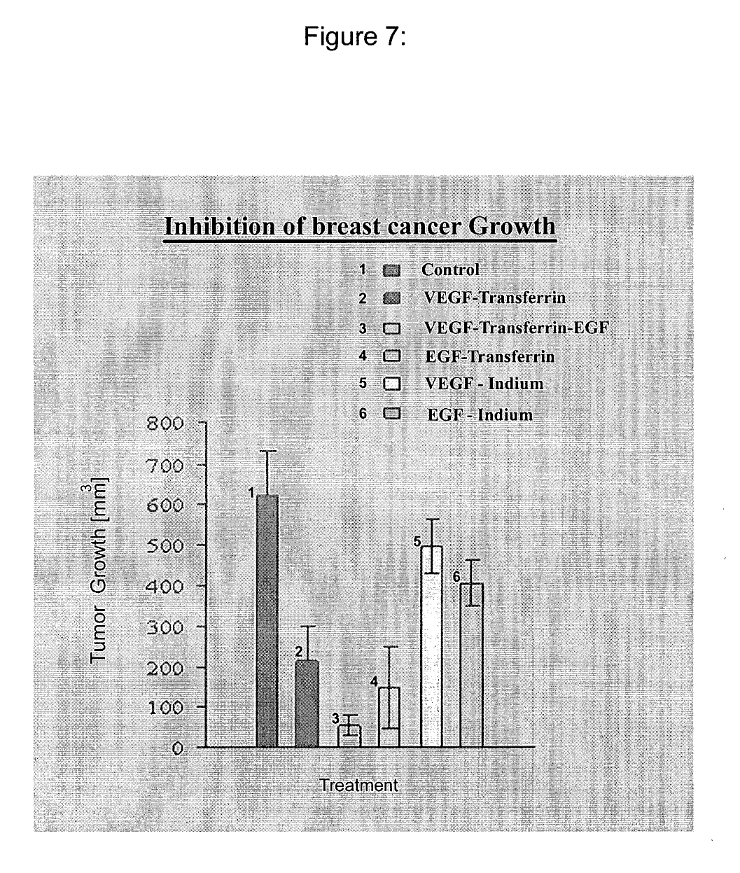

SCID mice were implanted with BCBM (JJ5). Control (Group 1) BCBM-SCID mice (n=6) were treated intraperitoneally with 25 nmol of unlabeled EGF or VEGF and ¹¹¹In TF-SA (200 uCi) once a week for 5 weeks. Experimental group 2 BCBM-SCID mice (n=6) were treated intraperitoneally with VEGF-¹¹¹In labeled transferrin (200 uCi) once a week for 5 weeks. Experimental group 3 BCBM-SCID mice (n=6) were treated intraperitoneally with VEGF-¹¹¹In labeled transferrin-EGF (200 uCi) once a week for 5 weeks. Experimental group 4 BCBM-SCID mice (n=6) were treated intraperitoneally with EGF-¹¹¹In labeled transferrin (200 uCi) once a week for 5 weeks. Experimental group 5 BCBM-SCID mice (n=6) were treated intraperitoneally with ¹¹¹In labeled VEGF (200 uCi) once a week for 5 weeks. Experimental group 6 BCBM-SCID mice (n=6) were treated intraperitoneally with ¹¹¹In labeled EGF (200 uCi) once a week for 5 weeks. At the end of the experiment the BCBM were resected from control and experimental mice and tumor weight and volume determined. Maximum inhibition of tumor growth is obtained by targeting the tumor cells and the tumor blood vessels with a single compound. The results of this experiment are shown in the bar graph of FIG. 7. In the bar graph presented by FIG. 7, bar #1 represents the tumor volume seen in control mice, bar #2 represents the tumor volume seen in mice administered VEGF-¹¹¹In-labeled transferrin compounds, bar #3 represents the tumor volume seen in mice administered VEGF-¹¹¹In-labeled transferrin-EGF compounds (the composition of the instant invention), bar #4 represents the tumor volume seen in mice administered EGF-¹¹¹In-labeled transferrin, bar #5 represents the tumor volume seen in mice administered ¹¹¹In-labeled VEGF and bar #6 represents the tumor volume seen in mice administered ¹¹¹In-labeled EGF. The P values representing the statistical significance of inhibition of tumor growth as compared with the tumor growth of the control are as follows: bar #3 0.0054; bar #2 0.0202; bar #4 0.0129; bar #5 0.3610 and bar #6 0.1328. A maximum, statistically significant amount of inhibition of tumor growth (BCBM) was seen in mice treated with the composition of the instant invention (VEGF-¹¹¹In-labeled transferrin-EGF compounds; shown by bar #3 in FIG. 7).

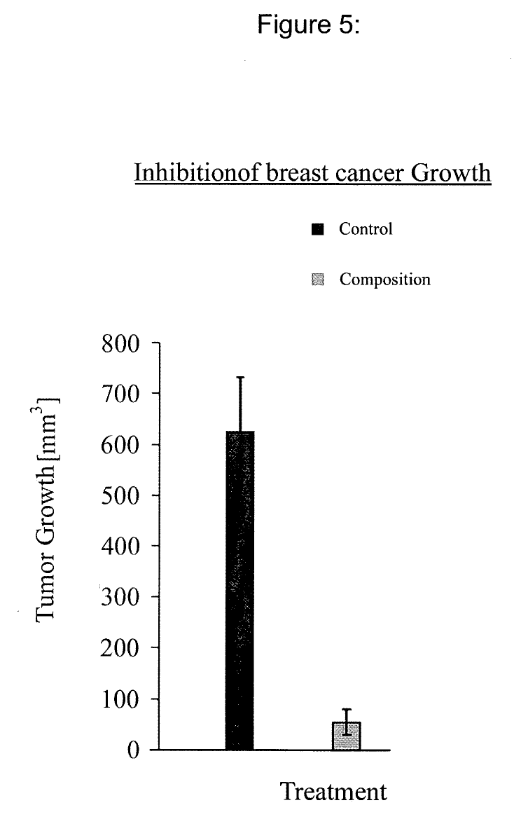

FIG. 5 shows a bar graph similar to the bar graph of FIG. 7 comparing the inhibition of breast cancer growth achieved by treatment with the composition of the instant invention (VEGF-¹¹¹In-labeled transferrin-EGF compounds). In the bar graph presented by FIG. 5, bar #1 represents the tumor volume seen in control mice and bar #2 represents the tumor volume seen in mice administered the composition of the instant invention (VEGF-¹¹¹In-labeled transferrin-EGF).

Animal Imaging

SCID mice (n=12) were implanted with BCBM (JJ5). Experimental group 1 BCBM-SCID mice (n=6) were treated intravenously (by injection into the tail vein) with VEGF-¹¹¹In labeled transferrin-EGF compounds (200 uCi). Control BCBM-SCID mice (n=6) were treated intravenously with 25 nmol of unlabeled EGF or VEGF and ¹¹¹In TF-SA (200 uCi). Mice bearing subcutaneous xenografts were injected intravenously in the tail vein with VEGF-¹¹¹In labeled transferrin-EGF compounds (200 μCi). For imaging study, at 0, 1, 24 and 48 hours post-injection, dynamic posterior whole-body images were obtained in groups of three to five mice on a Elscint SP4 gamma camera fitted with a medium energy pinhole collimator. Collimator-to-mouse distance was approximately 6 cm, with matrix size 256×256 byte. Number of counts on the images ranging from 20,000 to 100,000 over a time of 600 to 1200s. For biodistribution study, the mice were killed by cervical dislocation at A8 hours postinjection and the tumor plus samples of normal tissues (including blood, heart, lung, liver, kidney, spleen, stomach, intestines, muscle and brain) were removed to measure levels of radioactivity. Tissue samples were weighed and counted along with a standard of the injectate in a gamma counter (Packard Auto Gamma 5650, Packard Instruments, Downer's Grove, Ill.) using a window of 150±270 keV for ¹¹¹In. The tumor and normal tissue uptake of the compounds was expressed as percent injected dose per gram of tissue (% ID/g) and as tumor: normal tissue ratios.

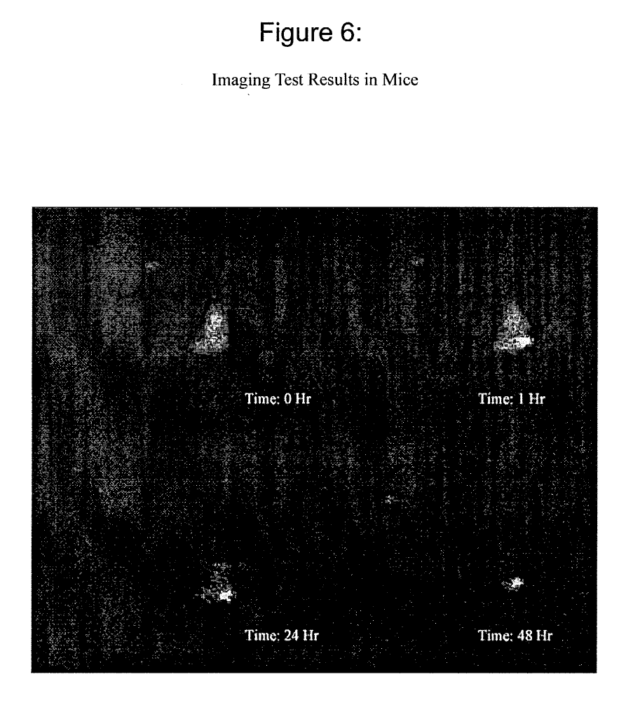

BCBM JJ5 was used in the imaging studies since it was shown to produce the highest tumor volumes in SCID mice (see FIG. 2). BCBM JJ5 has tumor cells that express high levels of EGF receptor and the blood vessels in the tumor are of human origin and thus express human VEGF receptors. VEGF-¹¹¹In-labeled transferrin-EGF (the composition of the instant invention ) was used to image JJ5 tumors. This radioactive compound targets EGF receptors on the tumor cells, VEGF receptors on the tumor blood vessels and transferrin receptors on both the tumor cells and tumor blood vessels. This multi-targeting approach provided images of the tumor. When constructs targeting either the EGF receptor or the VEGF receptor were used alone, no imaging of the JJ5 tumors was observed. FIG. 6 shows imaging test results obtained in mice. The image at the top left represents radioactive distribution in a mouse at 0 hours after injection, the image at the top right represents radioactive distribution in a mouse at 1 hour after injection, the image at the bottom left represents radioactive distribution in a mouse at 24 hours after injection and the image at the bottom right represents radioactive distribution localized in the tumor only 48 hours after injection. As illustrated in FIG. 6, the radiolabeled compound collected in several areas of the body but mostly in tumor site as this site is visualized as “hotspots”. This radiolabeled compound does not have any toxicity in the mice and the tumor imaging results show that the radiopharmaceutical has great potential for in vivo localization and biological characterization of cancers.

In summary, the composition of the instant invention is able to act as both a multi-targeting visualizing agent and a multi-targeting therapeutic agent. As is evidenced by the experimental examples described and shown herein, the instant invention provides a composition comprising a single non-immunogenic compound that can be used to target both the tumor cells and the endothelial cells of the tumor vasculature. Thus, the instant invention provides a novel multi-targeted approach to cancer diagnostics, imaging and therapy.

All patents and publications mentioned in this specification are indicative of the levels of those skilled in the art to which the instant invention pertains. All patents and publications are herein incorporated by reference to the same extent as if each individual patent and publication was specifically and individually indicated to be incorporated by reference.

It is to be understood that while a certain form of the invention is illustrated, it is not to be limited to the specific form or arrangement of parts herein described and shown. It will be apparent to those skilled in the art that various changes may be made without departing from the scope of the invention and the invention is not to be considered limited to what is shown and described in the specification.

One skilled in the art will readily appreciate that the present invention is well adapted to carry out the objects and obtain the ends and advantages mentioned, as well as those inherent therein. The oligonucleotides, peptides, polypeptides, biologically related compounds, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed various modifications of the described modes for carrying out the invention which are obvious to those skilled in the art are intended to be within the scope of the following claims.

What is claimed is:

1. A compound comprising human vascular endothelial growth factor (VEGF) and human epidermal growth factor (EGF) each operatively linked to radiolabeled human transferrin, wherein said human VEGF binds to human VEGF receptors on endothelial cell surfaces of intratumoral blood vessels, said human EGF binds to human EGF receptors on cell surfaces of tumor cells and said radiolabeled human transferrin binds human transferrin receptors on endothelial cell surfaces of intratumoral blood vessels and cell surfaces of tumor cells.

2. The compound in accordance with claim 1 wherein the radiolabel on said radiolabeled human transferrin is selected from the group comprising ¹¹¹In, ⁶⁷GA and ⁶⁸Ga.

3. The compound in accordance with claim 1 wherein the radiolabel on said radiolabeled human transferrin comprises ¹¹¹In.

4. A pharmaceutical composition comprising the compound of claim 1 and further including a pharmacologically effective amount of a carrier.

5. A pharmaceutical composition comprising the compound of claim 2 and further including a pharmacologically effective amount of a carrier.

6. A pharmaceutical composition comprising the compound of claim 3 and further including a pharmacologically effective amount of a carrier.

7. A conjugate consisting essentially of human vascular endothelial growth factor (VEGF) and human epidermal growth factor (EGF) each operatively linked to radiolabeled human transferrin, wherein said human VEGF binds to human VEGF receptors on endothelial cell surfaces of intratumoral blood vessels, said human EGF binds to human EGF receptors on cell surfaces of tumor cells and said radiolabeled human transferrin binds human transferrin receptors on endothelial cell surfaces of intratumoral blood vessels and cell surfaces of tumor cells.

8. The conjugate in accordance with claim 7 wherein the radiolabel on said radiolabeled human transferrin is selected from the group comprising ¹¹¹In, ⁶⁷Ga and ⁶⁸Ga.

9. The conjugate in accordance with claim 7 wherein the radiolabel on said radiolabeled human transferrin comprises ¹¹¹In.

10. A pharmaceutical composition comprising the conjugate of claim 7 and further including a pharmacologically effective amount of a carrier.

11. A pharmaceutical composition comprising the conjugate of claim 8 and further including a pharmacologically effective amount of a carrier.

12. A pharmaceutical composition comprising the compound of claim 9 and further including a pharmacologically effective amount of a carrier.


SEQUENCE LISTING (8 sequences)

SEQ ID NO: 1 - DNA, 495 bp, Homo sapiens
gcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaatccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcgg

SEQ ID NO: 2 - protein, 165 aa, Homo sapiens
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRCDKPRR

SEQ ID NO: 3 - DNA, 2037 bp, Homo sapiens
gtccctgataaaactgtgagatggtgtgcagtgtcggagcatgaggccactaagtgccagagtttccgcgaccatatgaaaagcgtcattccatccgatggtcccagtgttgcttgtgtgaagaaagcctcctaccttgattgcatcagggccattgcggcaaacgaagcggatgctgtgacactggatgcaggtttggtgtatgatgcttacttggctcccaataacctgaagcctgtggtggcagagttctatgggtcaaaagaggatccacagactttctattatgctgttgctgtggtgaagaaggatagtggcttccagatgaaccagcttcgaggcaagaagtcctgccacacgggtctaggcaggtccgctgggtggaacatccccataggcttactttactgtgacttacctgagccacgtaaacctcttgagaaagcagtggccaatttcttctcgggcagctgtgccccttgtgcggatgggacggacttcccccagctgtgtcaactgtgtccagggtgtggctgctccacccttaaccaatacttcggctactcgggagccttcaagtgtctgaaggatggtgctggggatgtggcctttgtcaagcactcgactatatttgagaacttggcaaacaaggctgacagggaccagtatgagctgctttgcctagacaacacccggaagccggtagatgaatacaaggactgccacttggcccaggtcccttctcataccgtcgtggcccgaagtatgggcggcaaggaggacttgatctgggagcttctcaaccaggcccaggaacattttggcaaagacaaatcaaaagaattccaactattcagctctcctcatgggaaggacctgctgtttaaggactctgcccacgggtttttaaaagtccccccaaggatggatgccaagatgtacctgggctatgagtatgtcactgccatccggaatctacgggaaggcacatgcccagaagccccaacagatgaatgcaagcctgtgaagtggtgtgcgctgagccaccacgagaggctcaagtgtgatgagtggagtgttaacagtgtagggaaaatagagtgtgtatcagcagagaccaccgaagactgcatcgccaagatcatgaatggagaagctgatgccatgagcttggatggagggtttgtctacatagcgggcaagtgtggtctggtgcctgtcttggcagaaaactacaataagagcgataattgtgaggatacaccagaggcagggtattttgctgtagcagtggtgaagaaatcagcttctgacctcacctgggacaatctgaaaggcaagaagtcctgccatacggcagttggcagaaccgctggctggaacatccccatgggcctgctctacaataagatcaaccactgcagatttgatgaatttttcagtgaaggttgtgcccctgggtctaagaaagactccagtctctgtaagctgtgtatgggctcaggcctaaacctgtgtgaacccaacaacaaagagggatactacggctacacaggcgctttcaggtgtctggttgagaagggagatgtggcctttgtgaaacaccagactgtcccacagaacactgggggaaaaaaccctgatccatgggctaagaatctgaatgaaaaagactatgagttgctgtgccttgatggtaccaggaaacctgtggaggagtatgcgaactgccacctggccagagccccgaatcacgctgtggtcacacggaaagataaggaagcttgcgtccacaagatattacgtcaacagcagcacctatttggaagcaacgtaactgactgctcgggcaacttttgtttgttccggtcggaaaccaaggaccttctgttcagagatgacacagtatgtttggccaaacttcatgacagaaacacatatgaaaaatacttaggagaagaatatgtcaaggctgttggtaacctgagaaaatgctccacctcatcactcctggaagcctgcactttccgtagacct

SEQ ID NO: 4 - protein, 679 aa, Homo sapiens
VPDKTVRWCAVSEHEATKCQSFRDHMKSVIPSDGPSVACVKKASYLDCIRAIAANEADAVTLDAGLVYDAYLAPNNLKPVVAEFYGSKEDPQTFYYAVAVVKKDSGFQMNQLRGKKSCHTGLGRSAGWNIPIGLLYCDLPEPRKPLEKAVANFFSGSCAPCADGTDFPQLCQLCPGCGCSTLNQYFGYSGAFKCLKDGAGDVAFVKHSTIFENLANKADRDQYELLCLDNTRKPVDEYKDCHLAQVPSHTVVARSMGGKEDLIWELLNQAQEHFGKDKSKEFQLFSSPHGKDLLFKDSAHGFLKVPPRMDAKMYLGYEYVTAIRNLREGTCPEAPTDECKPVKWCALSHHERLKCDEWSVNSVGKIECVSAETTEDCIAKIMNGEADAMSLDGGFVYIAGKCGLVPVLAENYNKSDNCEDTPEAGYFAVAVVKKSASDLTWDNLKGKKSCHTAVGRTAGWNIPMGLLYNKINHCRFDEFFSEGCAPGSKKDSSLCKLCMGSGLNLCEPNNKEGYYGYTGAFRCLVEKGDVAFVKHQTVPQNTGGKNPDPWAKNLNEKDYELLCLDGTRKPVEEYANCHLARAPNHAVVTRKDKEACVHKILRQQQHLFGSNVTDCSGNFCLFRSETKDLLFRDDTVCLAKLHDRNTYEKYLGEEYVKAVGNLRKCSTSSLLEACTFRRP

SEQ ID NO: 5 - DNA, 159 bp, Homo sapiens
aactctgattccgaatgcccgctgtctcatgacggttactgcctgcatgatggcgtatgcatgtacatcgaagctctggacaaatacgcatgcaactgtgttgtaggttacatcggcgaacgttgccagtatcgcgacctgaaatggtgggaactgcgt

SEQ ID NO: 6 - protein, 53 aa, Homo sapiens
NSDSECPLSHDGYCLHDGVCMYIEALDKYACNCVVGYIGERCQYRDLKWWELR

SEQ ID NO: 7 - DNA, 45 bp, Artificial
  sequence codes for a polylinker
ggtggcggtggctcgggcggtggtgggtcgggtggcggcggatct

SEQ ID NO: 8 - protein, 15 aa, Artificial
  sequence of a polylinker
GGGGSGGGGSGGGGS